(19) United States
(12) Patent Application Publication
(10) Pub. No.: US 2025/0282739 A1
(43) Pub. Date: Sep. 11, 2025

(54) PROCESS FOR THE PREPARATION OF TAFAMIDIS

(71) Applicant: Glenmark Life Sciences Limited, Mumbai (IN)

(72) Inventors: Venkata Raghavendra Charyulu PALLE, Pune (IN); Subbiah RAMAR, Vadodara (IN); Vishweshwar PEDDY, Hyderabad (IN); Santosh Ramesh BADGUJAR, Dombivali-West (IN); Vinayak Kacheshwar BHUJADE, Ahmednagar (IN); Yogesh Eknath GAGARE, Dombivali (IN); Suresh Babu NARAYANAN, Dombivli East (IN); Premchand Bansilal PATIL, Dombivli East (IN)

(21) Appl. No.: 18/857,532

(22) PCT Filed: Apr. 19, 2023
(86) PCT No.: PCT/IB2023/053997

§ 371 Date: Oct. 17, 2024

(30) Foreign Application Priority Data
Apr. 20, 2022 (IN) 202221023239

(51) Int. Cl. C07D 263/57

(52) U.S. Cl. CPC C07D 263/57

(57) ABSTRACT

The present invention relates to a process for the preparation of crystalline Form 4 of tafamidis from tafamidis meglumine salt.

### PRIORITY

This application claims the benefit of Indian Provisional Application No. 20/222,1023239 filed on Apr. 20, 2022, entitled “PROCESS FOR THE PREPARATION OF TAFAMIDIS”, the contents of which are incorporated herein by reference.

### BACKGROUND OF THE INVENTION

### Technical Field

The present invention relates to a process for the preparation of crystalline Form 4 of tafamidis from tafamidis meglumine salt.

### Description of the Related Art

Tafamidis, also known as, 2-(3,5-dichlorophenyl)-1,3-benzoxazole-6-carboxylic acid is represented by the structure of formula II.

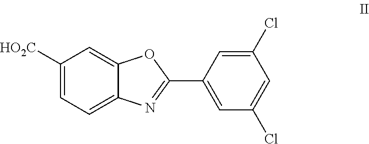 [SMILES: O=C(O)c1ccc2nc(-c3cc(Cl)cc(Cl)c3)oc2c1]

Tafamidis Meglumine, also known as, 2-(3,5-dichlorophenyl)-1,3-benzoxazole-6-carboxylic acid mono (1-deoxy-1-methylamino-D-glucitol) is represented by the structure of formula I,

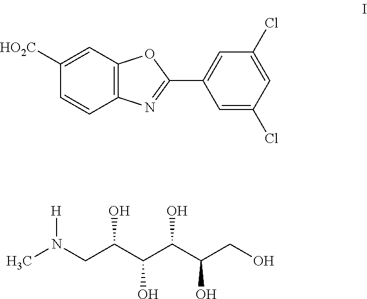 [SMILES: O=C(O)c1ccc2nc(-c3cc(Cl)cc(Cl)c3)oc2c1 ; CNC[C@H](O)[C@@H](O)[C@H](O)[C@H](O)CO]

Tafamidis (Vynmax®) and/or its meglumine salt (Vyndaqel®) are approved in the US, Europe and a few other countries, as an effective and safe oral medication for the treatment of the cardiomyopathy of transthyretin-mediated amyloidosis in adults to reduce cardiovascular mortality and cardiovascular-related hospitalization.

Tafamidis is disclosed in International Publication No. WO 2004/056315A2.

The synthesis of tafamidis is described in International Publication No. WO 2004/056315A2 and International Publication No. WO 2016/038500A1.

The object of the present invention is to provide an improved process for the preparation of crystalline Form 4 of tafamidis.

### SUMMARY OF THE INVENTION

The present invention provides a process for the preparation of crystalline Form 4 of tafamidis, comprising the steps of:
(a) providing a solution of tafamidis meglumine in a solvent selected from the group consisting of (C₁-C₄) amides, (C₂-C₃) sulfoxides, (C₅-C₆) pyrrolidones, and a mixture thereof;
(b) reacting the solution of the step (a) with an acid to form a reaction mixture; and
(c) obtaining crystalline Form 4 of tafamidis from the reaction mixture of the step (b) by combining the reaction mixture with a second solvent selected from the group consisting of (C₁-C₈) alcohols, (C₃-C₁₀) ketones, (C₁-C₃) nitriles, water and a mixture thereof.

### BRIEF DESCRIPTION OF THE DRAWINGS

FIG. 1 is a characteristic XRPD of crystalline Form 4 of tafamidis as obtained in Example 4.

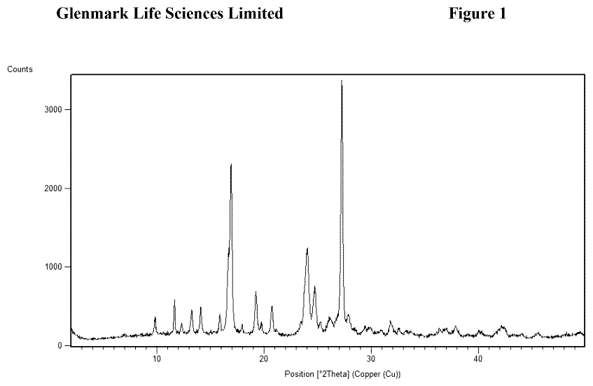

### DETAILED DESCRIPTION OF THE INVENTION

In one aspect, the present invention provides a process for the preparation of crystalline Form 4 of tafamidis, comprising the steps of:
(a) providing a solution of tafamidis meglumine in a solvent selected from the group consisting of (C₁-C₄) amides, (C₂-C₃) sulfoxides, (C₅-C₆) pyrrolidones, and a mixture thereof;
(b) reacting the solution of the step (a) with an acid to form a reaction mixture; and
(c) obtaining crystalline Form 4 of tafamidis from the reaction mixture of the step (b) by combining the reaction mixture with a second solvent selected from the group consisting of (C₁-C₈) alcohols, (C₃-C₁₀) ketones, (C₁-C₃) nitriles, water and a mixture thereof.

In the context of the present invention, the term “room temperature” means a temperature of about 25° C. to about 30° C.

In one embodiment, as used herein, the term “about” refers to any value which lies within the range defined by a number up to 10% of the value.

In one embodiment, in the step (a), the solvent is selected form the group consisting of a (C₁-C₄) amide such as dimethylformamide, dimethylacetamide, and the like; a (C₂-C₃) sulfoxide such as dimethyl sulfoxide and the like; a (C₅-C₆) pyrrolidone solvent such as N-Methyl-2-pyrrolidone and the like; and a mixture thereof.

In one embodiment, the step (a) may be carried out by stirring a solution of tafamidis meglumine at a temperature from about 20° C. to about 80° C. The stirring may be continued for any desired time period to achieve a complete dissolution of tafamidis meglumine. The solution may be optionally filtered to get a particle-free solution.

In one embodiment, in the step (b), the acid is selected from an organic acid or an inorganic acid.

In one embodiment, in the step (b), the organic acid is selected from the group consisting of a carboxylic acid, a sulfonic acid, and a mixture thereof.

In one embodiment, the carboxylic acid is selected from the group consisting of formic acid, acetic acid, propionic acid, trifluoroacetic acid and a mixture thereof.

In one embodiment, the sulfonic acid is selected from the group consisting of p-toluenesulfonic acid, ethane-1,2-disulfonic acid, ethanesulfonic acid, methanesulfonic acid, benzenesulfonic acid, and a mixture thereof.

In one embodiment, in the step (b), the inorganic acid is selected from the group consisting of hydrochloric acid, hydrobromic acid, sulfuric acid, nitric acid, phosphoric acid, and the like.

In one embodiment, the step (b) may be carried out by stirring the solution of the step (a) with an acid at a temperature from about 0° C. to about 40° C. The stirring may be continued for about 30 minutes to about 3 hrs or longer.

In one embodiment, in the step (c), the term “combining” means adding the reaction mixture of the step (b) to a second solvent, or adding the second solvent to the reaction mixture of the step (b).

In one embodiment, in the step (c), the second solvent is selected from the group consisting of an (C₁-C₈) alcohol such as methanol, ethanol, 1-propanol, 2-propanol, 1-butanol, 2-butanol, 1-pentanol and the like; a (C₃-C₁₀) ketone such as acetone, methyl isobutyl ketone, ethyl methyl ketone and the like; a (C₁-C₃) nitrile such as acetonitrile, propionitrile and the like; water; and a mixture thereof.

In one embodiment, the step (c) may be carried out at a temperature from about 10° C. to about 30° C.

In one embodiment, the step (c) may be carried out at a temperature from about 15° C. to about 20° C.

In an embodiment, the present invention provides a process for the preparation of crystalline Form 4 of tafamidis, comprising the steps of:
(a) providing a solution of tafamidis meglumine in a solvent selected from the group consisting of (C₁-C₄) amides, (C₂-C₃) sulfoxides, (C₅-C₆) pyrrolidones, and a mixture thereof;
(b) reacting the solution of the step (a) with acetic acid to form a reaction mixture; and
(c) obtaining crystalline Form 4 of tafamidis from the reaction mixture of the step (b) by combining the reaction mixture with a second solvent selected from the group consisting of (C₁-C₈) alcohols, (C₃-C₁₀) ketones, (C₁-C₃) nitriles, water and a mixture thereof.

In an embodiment, the present invention provides a process for the preparation of crystalline Form 4 of tafamidis, comprising the steps of:
(a) providing a solution of tafamidis meglumine in a solvent selected from the group consisting of (C₁-C₄) amides, (C₂-C₃) sulfoxides, and a mixture thereof;
(b) reacting the solution of the step (a) with an acid to form a reaction mixture; and
(c) obtaining crystalline Form 4 of tafamidis from the reaction mixture of the step (b) by combining the reaction mixture with a second solvent selected from the group consisting of (C₁-C₈) alcohols, water and a mixture thereof.

In an embodiment, the present invention provides a process for the preparation of crystalline Form 4 of tafamidis, comprising the steps of:
(a) providing a solution of tafamidis meglumine in a solvent selected from the group consisting of (C₁-C₄) amides such as dimethylformamide, dimethylacetamide; (C₂-C₃) sulfoxides such as dimethylsulfoxide, and a mixture thereof;
(b) reacting the solution of the step (a) with an acid to form a reaction mixture; and
(c) obtaining crystalline Form 4 of tafamidis from the reaction mixture of the step (b) by combining the reaction mixture with a second solvent selected from the group consisting of (C₁-C₈) alcohols such as methanol, ethanol, isopropanol; water and a mixture thereof.

Crystalline Form 4 of tafamidis is described in International Publication No. WO 2016/038500A1, having a X-ray powder diffraction pattern containing specific peaks at 15.9, 16.9, 18.0, 24.1 and 27.3±0.2 degrees 2 theta.

X-ray powder diffraction pattern of the crystalline Form 4 of tafamidis obtained by the process of the present invention substantially matches with the crystalline Form 4 of tafamidis disclosed in the International Publication No. WO 2016/038500A1.

In one embodiment, the crystalline Form 4 of tafamidis obtained by the process of the present invention is substantially free of the other polymorphs of tafamidis reported in the art.

In one embodiment, the term “substantially free” means the amount referred to is in no detectable quantity.

In the context of the present invention relating to the process for the preparation of crystalline Form 4 of tafamidis, the term “no detectable quantity” refers to other polymorphs of tafamidis in an amount of less than about 0.5% w/w. Preferably, less than about 0.1% w/w, still more preferably, absent.

In one embodiment, the crystalline Form 4 of tafamidis obtained by the process of the present invention is stable.

As used herein, the term “stable” refers to crystalline Form 4 of tafamidis which retains its original polymorphic form without undergoing polymorphic conversion over time.

In one embodiment, the stable crystalline Form 4 of tafamidis obtained by the process as described herein above has no detectable quantity of other polymorphs of tafamidis on storage.

In one embodiment, the stable Form 4 of tafamidis obtained by the process as described herein above has no detectable quantity of other polymorphs of tafamidis at room temperature or under accelerated stability conditions for extended periods of time.

In one embodiment, the present invention provides a process wherein tafamidis is obtained in a purity of ≥99.0% and wherein the level of the impurities designated herein as impurity A, impurity B, impurity C, or impurity D represented by the following structural formulae is less than 0.15% w/w relative to the amount of tafamidis, as determined by High Performance Liquid Chromatography (HPLC).

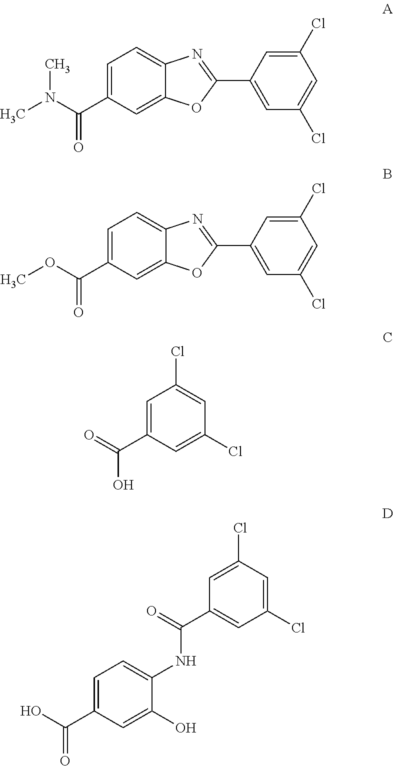 [SMILES: CN(C)C(=O)c1ccc2nc(-c3cc(Cl)cc(Cl)c3)oc2c1 ; COC(=O)c1ccc2nc(-c3cc(Cl)cc(Cl)c3)oc2c1 ; O=C(O)c1cc(Cl)cc(Cl)c1 ; O=C(O)c1ccc(NC(=O)c2cc(Cl)cc(Cl)c2)c(O)c1]

In one embodiment, the tafamidis is obtained in a purity of ≥99.0% and wherein the level any of the impurity as described above is less than 0.10%.

In one embodiment, the tafamidis is obtained in a purity of ≥99.0% and wherein the level any of the impurity as described above is less than 0.05%.

In one embodiment, the tafamidis is obtained in a purity of ≥99.0% and wherein the level any of the impurity as described above is not detected.

In one embodiment, the tafamidis is obtained in a purity of ≥99.5% and wherein the level of the impurity as described above is less than 0.15%.

In one embodiment, the tafamidis is obtained in a purity of ≥99.8% and wherein the level of the impurity as described above is less than 0.15%.

In one embodiment, the tafamidis meglumine used in the step (a) is prepared by the process, comprising the steps of:
(i) reacting 4-(3,5-dichlorobenzamido)-3-hydroxybenzoic acid with a coupling agent in a solvent to obtain a reaction mixture comprising tafamidis;
(ii) reacting the reaction mixture of the step (i) with meglumine to obtain a reaction mixture comprising tafamidis meglumine; and
(iii) separating the tafamidis meglumine from the reaction mixture of the step (ii).

In one embodiment, in the step (i), the coupling agent is selected from the group consisting of methanesulfonic acid, ethanesulfonic acid, p-toluenesulfonic acid, benzenesulfonic acid, camphorsulfonic acid, D-camphorsulfonic acid, L-camphorsulfonic acid, pyridinesulfonic acid, polyphosphoric acid, acetic anhydride, triflic anhydride; and a mixture thereof.

In one embodiment, in the step (i), the coupling agent used is methanesulfonic acid, ethanesulfonic acid, p-toluenesulfonic acid, benzenesulfonic acid, camphorsulfonic acid, D-camphorsulfonic acid, L-camphorsulfonic acid, or pyridinesulfonic acid.

In one embodiment, in the step (i), the coupling agent used is methanesulfonic acid.

In one embodiment, the step (i) is carried out in the presence of a solvent.

In one embodiment, in the step (i), the solvent is selected from the group consisting of a (C₁-C₄) amide such as dimethylformamide, dimethylacetamide and the like; a hydrocarbon such as toluene, xylene, chlorobenzene and the like; a sulfoxide such as dimethyl sulfoxide; N-methyl pyrrolidone; an alcohol such as methanol, ethanol, 1-propanol, 2-propanol, 1-butanol, 2-butanol, 1-pentanol and the like; a ketone such as acetone, methyl isobutyl ketone, ethyl methyl ketone and the like; a nitrile solvent such as acetonitrile, propionitrile and the like; an ether such as tetrahydrofuran, methyl tert-butyl ether and the like; water; and a mixture thereof.

In one embodiment, in the step (i), the solvent used is (C₁-C₄) amide solvent.

In one embodiment, in the step (i), the amide solvent used is dimethylacetamide or dimethylformamide.

In one embodiment, in the step (i), the amide solvent used is dimethylacetamide.

In one embodiment, the step (i) may be carried out by stirring the reaction mixture at a suitable temperature of about 0° C. to reflux temperature of the solvent used. The stirring time may range from about 30 minutes to about 26 hours, or longer.

In one embodiment, the step (i) may be carried out for a sufficient time for the complete cyclization of 4-(3,5-dichlorobenzamido)-3-hydroxybenzoic acid, and till the content of 4-(3,5-dichlorobenzamido)-3-hydroxybenzoic acid is not detected in the reaction mixture.

In one embodiment, the tafamidis obtained is not isolated from the reaction mixture and is reacted with meglumine to form the tafamidis meglumine.

As used herein, the term “not isolated” means tafamidis is not separated as a solid.

In one embodiment, the step (ii) may be carried out by stirring the reaction mixture of the step (i) with meglumine at a temperature from about 10° C. to about 35° C. The stirring time may range from about 30 minutes to about 10 hours, or longer.

In one embodiment, the step (iii) is carried out by the process, comprising:
(iii-a) combining the reaction mixture of the step (ii) with a second solvent selected from the group consisting of (C₁-C₈) alcohols, (C₃-C₁₀) ketones, (C₁-C₃) nitriles, water and a mixture thereof; or
(iii-b) removing the solvent from the reaction mixture of the step (ii).

In one embodiment, in the step (iii-a), the term “combining” means adding the reaction mixture of the step (ii) to a second solvent, or adding the second solvent to the reaction mixture of the step (ii).

In one embodiment, in step (iii-a), the second solvent is selected from the group consisting a (C₁-C₈) alcohol such as methanol, ethanol, 1-propanol, 2-propanol, 1-butanol, 2-butanol, 1-pentanol and the like; a (C₃-C₁₀) ketone such as acetone, methyl isobutyl ketone, ethyl methyl ketone and the like; a (C₁-C₃) nitrile such as acetonitrile, propionitrile and the like; water; and a mixture thereof.

In one embodiment, in step (iii-b), the removal of solvent may be accomplished by substantially complete evaporation of the solvent; or concentrating the solution, or cooling the solution, if required and then filtering the resulting solid. The solution may also be completely evaporated in, for example, a rotavapor, a vacuum tray dryer or in a conventional reactor under vacuum above about 720 mm Hg. The drying may be performed in vacuum tray drier at a temperature of about 40° C. to about 70° C.

In one embodiment, tafamidis meglumine obtained by the above described process may be recrystallized from a solvent selected from the group consisting of alcohols such as methanol, ethanol, 1-propanol, 2-propanol, 1-butanol, 2-butanol, 1-pentanol and the like; water; and a mixture thereof.

In one embodiment, 4-(3,5-dichlorobenzamido)-3-hydroxybenzoic acid used in step (i) is prepared by a process, the process comprising the steps of:
(1) reacting 3,5-dichlorobenzoic acid with thionyl chloride in the presence of a solvent to obtain 3,5-dichlorobenzoyl chloride;
(2) reacting 3,5-dichlorobenzoyl chloride obtained from the step (1) with 4-amino-3-hydroxybenzoic acid in the presence of a solvent and a base to obtain 4-(3,5-dichlorobenzamido)-3-hydroxybenzoic acid.

In one embodiment, the solvent used in the step (1) and the step (2) is selected from the group consisting of an amide such as dimethylformamide, dimethylacetamide and the like; a hydrocarbon such as toluene, xylene, chlorobenzene and the like; a sulfoxide such as dimethyl sulfoxide; N-methyl pyrrolidone; an alcohol such as methanol, ethanol, 1-propanol, 2-propanol, 1-butanol, 2-butanol, 1-pentanol and the like; a ketone such as acetone, methyl isobutyl ketone, ethyl methyl ketone and the like; a nitrile such as acetonitrile, propionitrile and the like; water; and a mixture thereof.

In one embodiment, the step (1) may be carried out by stirring at a temperature from about 10° C. to about 90° C. The stirring time may range from about 1 hour to about 5 hours, or longer.

In embodiments, the step (2) may be carried out for sufficient time for the formation of 4-(3,5-dichlorobenzamido)-3-hydroxybenzoic acid and till the content of the starting materials are not detected in the reaction mixture.

In one embodiment, the step (2) may be carried out by stirring at a temperature from about 10° C. to about 40° C. The stirring time may range from about 30 minutes to about 5 hours, or longer.

In one embodiment, the present invention provides pharmaceutical compositions comprising tafamidis obtained by the process(es) herein described, having a D90 particle size of less than about 250 microns, preferably less than about 150 microns, more preferably less than about 50 microns, still more preferably less than about 20 microns, still more preferably less than about 15 microns, and most preferably less than about 10 microns.

In one embodiment, the present invention provides pharmaceutical compositions comprising tafamidis obtained by the process(es) herein described, having a D50 particle size of less than about 250 microns, preferably less than about 150 microns, more preferably less than about 50 microns, still more preferably less than about 20 microns, still more preferably less than about 15 microns, and most preferably less than about 10 microns.

The particle size for tafamidis disclosed herein above can be obtained by, for example, any milling, grinding, micronizing or other particle size reduction method known in the art to bring the solid state tafamidis into any of the foregoing desired particle size range.

The examples that follow are provided to enable one skilled in the art to practice the invention and are merely illustrative of the invention. The examples should not be read as limiting the scope of the invention as defined in the features and advantages.

### EXAMPLES

### Example 1: Preparation of 4-(3,5-dichlorobenzamido)-3-hydroxybenzoic acid

3,5-Dichlorobenzoic acid (16.21 g) was charged in toluene (80 mL) and dimethyl formamide (0.38 g) was added under nitrogen atmosphere. Thionyl chloride (13.2 g) was added to the reaction mass at about 20-30° C., and heated to about 60-70° C. for about 2 h. The reaction mass was cooled to about 45-50° C. and toluene was distilled off by vacuum to obtain an oily material. The oily material was stripped out by toluene (20 mL) and degassed for about 1 hr at about 60-65° C. The obtained oil (3,5-dichlorobenzoyl chloride) was cooled at about 25-30° C., and dissolved in tetrahydrofuran (20 mL). 4-Amino-3-hydroxybenzoic acid (10 g) was dissolved in a mixture of tetrahydrofuran (100 mL) and water (19 mL). To this solution was added the above prepared solution of 3,5-dichlorobenzoyl chloride in tetrahydrofuran at about 15-20° C., and stirred for about 30 min. To this reaction mixture, trimethylamine (7.9 g) was added and heated to about 35-40° C. and stirred for about 90 min. After completion of the reaction, the reaction mass was cooled to about 25-30° C., added water and ethanol to obtain the slurry, stirred for about 60 min at about 20-30° C. and filtered. The obtained solid was washed twice with ethanol, dried in vacuum tray dryer at about 50-60° C. to obtain title compound (Yield: 84.83%).

### Example 2: Preparation of Tafamidis Meglumine

4-(3,5-dichlorobenzamido)-3-hydroxybenzoic acid (10 g) was added in solvent mixture of dimethylacetamide (20 mL) and toluene (180 mL). To this mixture was added triethylamine (3.4 g), and stirred for about 1 hr at about 25-30° C. Methane sulfonic acid (4.71 g) was added and the reaction mass was refluxed for about 24 hr at about 110-120° C. After completion of reaction, the reaction mass was cooled to about 60-70° C. Activated charcoal (1 g) and dimethyl acetamide (10 mL) were added to the reaction mixture, stirred for about 15-20 min, cooled to about 45-55° C., filtered in tetrahydrofuran through hyflo and washed with tetrahydrofuran. The filtrate was cooled to about 25-30° C., added water, stirred and organic layer separated. Dimethylacetamide (50 mL) and D-meglumine (5.98 g) were added in the organic layer, stirred for about 10-15 min, added solvent mixture of iso-propyl alcohol (80 mL) and water (20 mL) and stirred for 1 hr. The product was filtered and washed with iso-propyl alcohol and dried in vacuum tray dryer at about 55-60° C. to obtain crude tafamidis meglumine. (Yield: 84.41%)

### Example 3: Preparation of Tafamidis Meglumine

Tafamidis meglumine crude (7.5 g) was added in a solvent mixture of iso-propyl alcohol (90 mL) and water (27 mL) and the reaction mixture was heated at about 75-80° C. to obtain a clear solution. The reaction mixture was stirred for about 10-15 min, cooled to about 65-70° C., filtered to remove any insoluble particle and washed with hot solvent mixture of iso-propyl alcohol (6 mL) and water (1.5 mL). The filtrate was heated at about 75-80° C., stirred for about 5-10 min, cooled to about 55-57° C., added tafamidis meglumine as seed (0.08 g). The reaction mass was further cooled to about 10-15° C., stirred for about 60-70 min and filtered. The obtained solid was washed with iso-propyl alcohol and dried in vacuum tray dryer at about 55-60° C. to obtain Tafamidis meglumine (Yield: 90.0%).

### Example 4: Preparation of Tafamidis Form-4

Tafamidis Meglumine salt (3 g) was dissolved in dimethyl sulfoxide (33 mL) at about 50-60° C. The clear solution was filtered through hyflo for obtaining particle free solution, and washed with hot dimethyl sulfoxide (3 mL). The clear solution was cooled to about 25-30° C., and added acetic acid (0.4 g), the clear reaction mass was stirred for about 60-70 min at about 25-30° C. The clear reaction mass was added to a solvent mixture of iso-propyl alcohol (48 mL) and water (72 mL) at about 15-20° C. The solid was stirred for about 1 hr, filtered and washed with a mixture of iso-propyl alcohol and water, suck dried under vacuum. The obtained solid was dried in Air Tray Drier (ATD) for about 20 hr at about 50-80° C. to obtain the title compound. Yield: 1.47 g (80%). Purity: 99.75%. The crystalline Form 4 of tafamidis has no detectable quantity of Form 1 as determined by absence of characteristic peak reflections at about 20.2±0.2 degrees 2 theta. Stability data of crystalline Form 4 of tafamidis prepared by the present invention at periodic intervals:

| Storage | Polymorphic | Time | | | |
|---|---|---|---|---|---|
| Conditions | form | 15 days | 1 month | 2 months | 3 months |
| 25° C. | Form 1 | ND | ND | ND | ND |
| 40° C. | | ND | ND | ND | ND |
| at 75% RH | | | | | |

ND—not detected

### Example 5: Preparation of Tafamidis Form-4

Tafamidis (1 g) was dissolved in dimethyl sulfoxide (19 mL) at about 50-60° C. The clear solution was filtered through hyflo for particle free solution and washed with hot dimethyl sulfoxide. The clear solution was cooled to about 25-30° C. and added to water (60 mL) at about 15-20° C. The solid was stirred for about 1 hr, filtered, washed with water and suck dried under vacuum. The obtained solid was dried in Air Tray Drier (ATD) for about 20 hr at about 50-80° C. to obtain title compound. Yield: 0.92 g (92%).

### Example 6: Preparation of Tafamidis Form-4

Tafamidis Meglumine salt (5 g) was dissolved in dimethyl sulfoxide (60 mL) at about 50-60° C. The clear solution was filtered through hyflo for particle free solution. The clear solution was cooled to about 25-30° C., and added to mixture of iso-propyl alcohol (80 mL), water (120 mL) and acetic acid (0.65 g) at about 15-20° C. The slurry was stirred for about 1 hr, filtered and washed with a mixture of iso-propyl alcohol and water, suck dried under vacuum. The obtained solid was dried in Air Tray Drier (ATD) for about 12 hr at about 50-55° C. to obtain title compound. Yield: 2 g (65%).

### Example 7: Preparation of Tafamidis Form-4

Tafamidis Meglumine salt (5 g) was dissolved in dimethyl sulfoxide (60 mL) at about 50-60° C. The clear solution was filtered through hyflo for particle free solution. The clear solution was cooled to about 25-30° C., and added to mixture of iso-propyl alcohol (80 mL), water (120 mL) and methane sulphonic acid (1.15 g) at about 15-20° C. The slurry was stirred for about 1 hr, filtered and washed with a mixture of iso-propyl alcohol and water, suck dried under vacuum. The obtained solid was dried in Air Tray Drier (ATD) for about 12 hr at about 50-55° C. to obtain title compound. Yield: 2.4 g (78%).

### Example 8: Preparation of Tafamidis Form-4

Tafamidis Meglumine salt (5 g) was dissolved in dimethyl sulfoxide (60 mL) at about 50-60° C. The clear solution was filtered through hyflo for particle free solution. The clear solution was cooled to about 25-30° C., and added to mixture of iso-propyl alcohol (80 mL), water (120 mL) and concentrated hydrochloric acid (1.3 g) at about 15-20° C. The slurry was stirred for about 1 hr, filtered and washed with a mixture of iso-propyl alcohol and water, suck dried under vacuum. The obtained solid was dried in Air Tray Drier (ATD) for about 12 hr at about 50-55° C. to obtain title compound. Yield: 2.2 g (71%).

1. A process for the preparation of crystalline Form 4 of tafamidis, comprising the steps of:
(a) providing a solution of tafamidis meglumine in a (C₂-C₃) sulfoxide solvent;
(b) reacting the solution of the step (a) with an acid to form a reaction mixture; and
(c) obtaining crystalline Form 4 of tafamidis from the reaction mixture of the step (b) by combining the reaction mixture with a second solvent selected from the group consisting of (C₁-C₈) alcohols, water and a mixture thereof.

2. The process according to claim 1, wherein the acid used in the step (b) is selected from the group consisting of an organic acid, an inorganic acid, and a mixture thereof.

3. The process according to claim 2, wherein the organic acid is selected from the group consisting of a carboxylic acid, a sulfonic acid, and a mixture thereof.

4. The process according to claim 3, wherein the carboxylic acid is selected from the group consisting of formic acid, acetic acid, propionic acid, trifluoroacetic acid and a mixture thereof.

5. The process according to claim 3, wherein the sulfonic acid is selected from the group consisting of p-toluenesulfonic acid, ethane-1,2-disulfonic acid, ethane sulfonic acid, methane sulfonic acid, benzene sulfonic acid, and a mixture thereof.

6. The process according to claim 2, wherein the inorganic acid is selected from the group consisting of hydrochloric acid, hydrobromic acid, sulfuric acid, nitric acid, phosphoric acid, and a mixture thereof.

7. The process according to claim 1, wherein the crystalline Form 4 of tafamidis contains less than about 0.5% w/w of crystalline Form 1 of tafamidis.

8. The process according to claim 1, wherein the tafamidis meglumine used in the step (a) is prepared by a process comprising the steps of:
(i) reacting 4-(3,5-dichlorobenzamido)-3-hydroxybenzoic acid with a coupling agent in a (C₁-C₄) amide solvent to obtain a reaction mixture comprising tafamidis;
(ii) reacting the reaction mixture of the step (i) with meglumine to obtain a reaction mixture comprising tafamidis meglumine; and
(iii) separating the tafamidis meglumine from the reaction mixture of the step (ii).

9. (canceled)

10. The process according to claim 8, wherein the amide solvent is dimethylacetamide or dimethylformamide.

11. The process according to claim 8, wherein the step (iii) comprises:
(iii-a) combining the reaction mixture of the step (ii) with a second solvent selected from the group consisting of (C₁-C₈) alcohols, water and mixtures thereof; or
(iii-b) removing the solvent from the reaction mixture of the step (ii).

12. The process according to claim 1, wherein the (C₂-C₃) sulfoxide solvent used in the step (a) is dimethyl sulfoxide.

13. The process according to claim 1, wherein the (C₁-C₈) alcohols in the step (c) is selected from the group consisting of methanol, ethanol, 1-propanol, 2-propanol, 1-butanol, 2-butanol, 1-pentanol, and a mixture thereof.
